(19) United States
(12) Patent Application Publication
(10) Pub. No.: US 2015/0184096 A1
(43) Pub. Date: Jul. 2, 2015

(54) METHOD FOR BIODIESEL GENERATION

(71) Applicant: National Cheng Kung University, Tainan City (TW)

(72) Inventors: Jiunn-Der LIAO, Tainan City (TW); Aharon GEDANKEN, Tainan City (TW); Teng-Chien CHEN, Kaohsiung City (TW)

(21) Appl. No.: 14/526,736

(22) Filed: Oct. 29, 2014

Related U.S. Application Data
(60) Provisional application No. 61/921,172, filed on Dec. 27, 2013.

(51) Int. Cl. C10L 1/02; C07C 67/02

(52) U.S. Cl. CPC C10L 1/026; C07C 67/02; C10L 2200/0476; C10L 2270/026; C10L 2290/36; C07C 2521/08; C07C 2523/02

(57) ABSTRACT

A method for biodiesel generation is disclosed, which comprising: providing a plurality of nano-particles each containing an alkali metal compound or an alkaline earth metal compound; forming a composite in which the nano-particles are adhered on a support; and performing a transesterification reaction by contacting the composite with a target.

### CROSS REFERENCE TO RELATED APPLICATION

This application claims the benefit of filing date of U.S. Provisional Application Ser. No. 61/921,172, entitled “A Detailed Description of the New Discovery on the “Acceleration of the Transesterification Reaction conducted with Nano-sized SrO Supported on Silica Spheres Under Microwave radiation”” filed on Dec. 27, 2013.

### BACKGROUND OF THE INVENTION

1. Field of the Invention

The present invention relates to a method for biodiesel generation, and especially to an easy, quick, and environmental-friendly method for biodiesel generation.

2. Description of Related Art

Biodiesel belongs to an alkyl ester-based fuel generated from the reaction between long chain fatty acid and alkyl alcohol in the presence of a catalyst; in which long chain fatty acid can be obtained from animal and plant fats or cooked oil. It is proved that the biodiesel is an effective fuel for diesel vehicle engines and the quality of engine exhausts can be improved. The biodiesel can be directly used as the energy source or the addictive to petroleum diesel without modifying the conventional fossil diesel engines. In addition, the biodiesel is much safer than the petroleum diesel due to it has higher cetane numbers and lower flash point than the petroleum diesel. For instance, data published by the Environmental Protection Agency (EPA) indicates that pure form biodiesel (B100) such as methyl soyate as well as mixture biodiesel (B20) containing 20% biodiesel and 80% petroleum diesel can be used in replace of diesel fuel, to reduce pollutant emission and improve ambient air quality.

Currently, techniques for conversing animal and plant fats or cooked oil into biodiesel include dilution, pyrolysis, micro-emulsification and transesterification; in which transesterification is the most effective manner. A pretreatment is required for cooked oil before the transesterification reaction of the cooked oil, comprising: filtering the cooked oil with a filter and then removing water contained in the filtered cooked oil by distillation. Furthermore, pre-esterification is required for free fatty acid contained in the cooked oil. However, lots of complex procedures for refining the cooked oil have to be performed; and the efficiency of the sequent transesterification reaction of the refined cooked oil may further be affected if the fined quality thereof is not controlled well during the procedures for refining the cooked oil. In addition, the transesterification reaction comprises the following steps: mixing low-carbon alcohol and basic catalyst; placing the mixture for a period of time until the alkyl ester layer (crude biodiesel) separated from the glycerol layer; repeating the transesterification reaction on the top-layered crude biodiesel; recycling methanol contained in the obtained crude biodiesel by distillation; neutralizing, washing, and removing water in the obtained crude biodiesel by distillation to obtain pure biodiesel. However, this method has disadvantages of low biodiesel conversion, complex operating procedures, long operating time, and high cost for equipment.

In the tendency towards environment protection and higher demand for biodiesel, it is desirable to provide an easy and quick method to generate biodiesel from cooked oil, leading great contribution to economy and environment protection.

### SUMMARY OF THE INVENTION

An object of the present invention is to provide a method for biodiesel generation, so that biodiesel can be easily and quickly obtained through a single procedure, and environment pollution can be avoided because no chemical substances such as strong acids and bases are needed in the method of the present invention.

To achieve the object, the method for biodiesel generation of the present invention comprises the following steps: providing a plurality of nano-particles each containing an alkali metal compound or an alkaline earth metal compound; forming a composite in which the nano-particles are adhered on a support; and performing a transesterification reaction by contacting the composite with a target.

In the present invention, a material of the nano-particle contains an alkali metal compound or an alkaline earth metal compound. The alkali metal compound includes lithium (Li), sodium (Na), potassium (K), rubidium (Rb), cesium (Cs), or francium (Fr) containing compound; and the alkaline earth metal compound includes beryllium (Be), magnesium (Mg), calcium (Ca), strontium (Sr), barium (Ba), or radium (Ra) containing compound. Also, the material of the nano-particle preferably contains an alkali metal oxide or an alkaline earth metal oxide; and more preferably contains an alkaline earth metal oxide such as barium oxide (BaO), strontium oxide (SrO), calcium oxide (CaO) or magnesium oxide (MgO). Referring to the catalytic activity of the alkaline earth metal oxide in the transesterification reaction, the order of catalytic activity among the alkaline earth metal oxide catalysts is BaO>SrO>CaO>MgO. Since strontium oxide (SrO) is difficult to be dissolved in vegetable oil, methanol, and fatty acid methylester, strontium oxide (SrO) can be easily separated from the biodiesel or by-product thereof after the transesterification reaction is completed. Therefore, strontium oxide (SrO) is most preferably contained in the nano-particle.

The size of the nano-particle is not particularly limited, and may be 20 nm to 1000 nm, preferably 100 nm to 500 nm, and more preferably 90 nm to 200 nm. Also, an amount of nano-particles adhered on a support may be 0.5 wt % to 10 wt %, and preferably 2 wt % to 5 wt % based on a total weight of nano-particles and the support. Those skilled in the art can consider the factors such as the energy source, the catalytic effect, or the catalytic surface area to appropriately select the size as well as the amount of nano-particles according to the practical applications. However, the present invention is not limited to the conditions of the examples disclosed below.

In the present invention, the kind of the support is not particularly limited and can be any common support in the related art, for example, a silica carrier or another silicate-based carrier. The size of the support is not particularly limited either and can be appropriately selected by a person skilled in the art in accordance with the practical requirements including the catalytic effect, the catalytic surface area, the energy source, and the size of the nano-particle. For example, the size of the support can be 1 mm to 30 mm, preferably 1 mm to 16 mm, and more preferably 1 mm to 3 mm. However, the present invention is not limited to the conditions of the examples disclosed below.

In the present invention, the transesterification reaction can be performed through a microwave (MW) radiation treatment, a radio frequency (RF) treatment, or a laser treatment. Both the power and the treatment time of the microwave radiation treatment, the radio frequency treatment, or the laser treatment are not limited. The power and the treatment time thereof can be easily adjusted by a person skilled in the art according to the treatment conditions such as the energy intensity, the temperature, the focus area and so on. Particularly, the treatment time for the transesterification reaction can be reduced when a treatment with high energy, high temperature accompanies with accurate focus area is performed, due to the energy centralized. In contrast, the treatment time for the transesterification reaction have to be elongated when a treatment with low energy, low temperature accompanied with broader focus area is performed. Otherwise, the transesterification reaction can be performed under a low power and low temperature heating condition when the focus area is highly accurate during the reaction, thus achieving the energy conservation (low energy-consumption). In particular, the treatment time can be 10 seconds to 3 minutes under the microwave radiation treatment with 800 W to 1200 W carried out at 60° C. or more; or the treatment time can be 10 seconds to 2 minutes under the microwave radiation treatment with 1000 W to 1200 W carried out at 60° C. to 80° C. As long as the requirement for the reactive degree of the transesterification reaction is completed, the transesterification reaction can be performed in a batch processing or continuous processing. In other words, as long as the temperature for proceeding the transesterification reaction can be achieved within a predetermined area of the target, any manner can be used in the present invention.

In the present invention, not only the nano-particle can be adhered on a support but also a part of the nano-particle is preferably embedded in the support. Therefore, the nano-particle is hard to be separated from the support, preventing the nano-particle from spreading into the atmosphere. The embedding manner for the nano-particle is not particularly limited.

For instance, the transesterification reaction is performed under a higher temperature to soften the support, and thus nano-particles are embedded into the support. Alternatively, a second compound can further be formed between the support and the part of nano-particles embedded in the support. For instance, strontium silicate (SrSiO₃) compound can be formed stably at an interface between the SrO nano-particle and the silica carrier while SrO nano-particles are embedded into the silica carrier. Apparently, the second compound can be varied with the kind of the nano-particle and the support.

Besides, the target in the present invention is not particularly limited, and any material containing triglycerides can be used without limitation; for example, soybean, cottonseed oil, cooked oil, and algae. Accordingly, the treatment time is varied with the kind of the target. The transesterification reaction can be completed in only about 40 seconds while soybean is used as the target; about 2 minutes while algae is used as the target; and about 3 minutes while cooked oil is used as the target under the microwave radiation treatment with 1200 W. The definition of reaction completed means the triglycerides contained in the target are completely converted into biodiesel, and the conversion percentages of the biodiesel related to the target can be reached to 90% or more.

Taiwanese dietary habit results in plenty of cooked oil every day. By using the method of the present application, cooked oil generated in families, restaurants, and schools can be recycled to produce biodiesel from triglycerides. The method of the present application has a great contribution towards economy and environmental protection. After the transesterification reaction catalyzed by the composite in which the nano-particle is adhered on the support, three layers are separated automatically, comprising: a top biodiesel layer, a bottom glycerol layer (by-product), and a SrO catalyst layer between them. In addition, the SrO catalyst in the SrO catalyst layer can be recycled to reuse in the next transesterification reaction, and both biodiesel and glycerol have economic benefits.

Compared to the well-known catalysts used in the related art, potassium hydroxide (KOH) and sodium hydroxide (NaOH) are dissolved in both the biodiesel layer and the glycerol layer after the transesterification reaction. Thus, additional isolation and purification processes are needed and those catalysts are hard to be recycled. Therefore, the method of the present application has the advantages of the simplified processes, the lower cost, and the improved purity of the final product. Furthermore, the catalyst to be recycled can be easily separated from the products and reused several times for another transesterification process. Besides, environment pollution can be avoided because no chemical substances such as strong acids and bases are needed in the method of the present invention, and the by-product also has its industrial applicability.

Other objects, advantages, and novel features of the invention will become more apparent from the following detailed description when taken in conjunction with the accompanying drawings.

### BRIEF DESCRIPTION OF THE DRAWINGS

FIG. 1 is an energy-dispersive X-ray spectroscopy (EDS) spectrum of the SrO nano-particles on the silica support according to one preferred embodiment of the present invention;

FIG. 2 is an X-ray diffraction (XRD) pattern of the SrO nano-particles on the silica support according to one preferred embodiment of the present invention;

FIG. 3 is a diagram showing a size distribution of the SrO nano-particles measured by dynamic light scattering (DLS) analysis according to one preferred embodiment of the present invention;

FIG. 4 is a nuclear magnetic resonance (NMR) spectrum of biodiesel according to one preferred embodiment of the present invention;

FIG. 5A is a result of the catalytic stability test for SrO nano-particles on the silica support according to one preferred embodiment of the present invention;

FIG. 5B is a result of the oil content (%) in the microalgae sample according to one preferred embodiment of the present invention;

FIG. 6A is a result of the biodiesel conversion according to another preferred embodiment of the present application; and

FIG. 6B is a result of the biodiesel conversion according to further another preferred embodiment of the present application.

### DETAILED DESCRIPTION OF THE PREFERRED EMBODIMENT

Hereinafter, exemplary examples of the present invention will be described in detail. However, the present invention is not limited to the examples disclosed below, but can be implemented in various forms. The following examples are described in order to enable those of ordinary skill in the art to embody and practice the present invention, and those skilled in the art will appreciate that various modifications, additions and substitutions are possible.

### Synthesis of SrO Nano-Particles

0.5 grams of strontium acetyl acetonate were added into a beaker containing 40 ml of benzyl alcohol. 1.5 grams of solid silica particles as supports having particle size about 1 mm were also added into the beaker. The mixture was heated in a domestic microwave oven (1200 W) for 10 minutes (min). The reaction was carried out under an argon atmosphere to avoid carbon dioxide (CO₂) molecules resulting in the formation of strontium carbonate (SrCO₃). After the reaction completed, the SrO nano-particles tightly adhered on the entire surfaces of the silica supports to form the composites of the present embodiment; the amount of the SrO nano-particles adhered on the supports was about 2 wt % to 5 wt % based on the total weight of the SrO nano-particles and the silica supports (the composite); and the SrO nano-particles have an average particle size about 100 nm according to the scanning electron microscope (SEM) image (figure not shown). FIG. 1 depicts the energy-dispersive X-ray spectroscopy (EDS) spectrum of the composites in which the SrO nano-particles are adhered on the silica supports. We can observe the presence of strontium (Sr) and oxygen (O). The presence of carbon (C) is resulted from the formation of SrCO₃ due to a little amount of CO₂ molecules participated in the reaction.

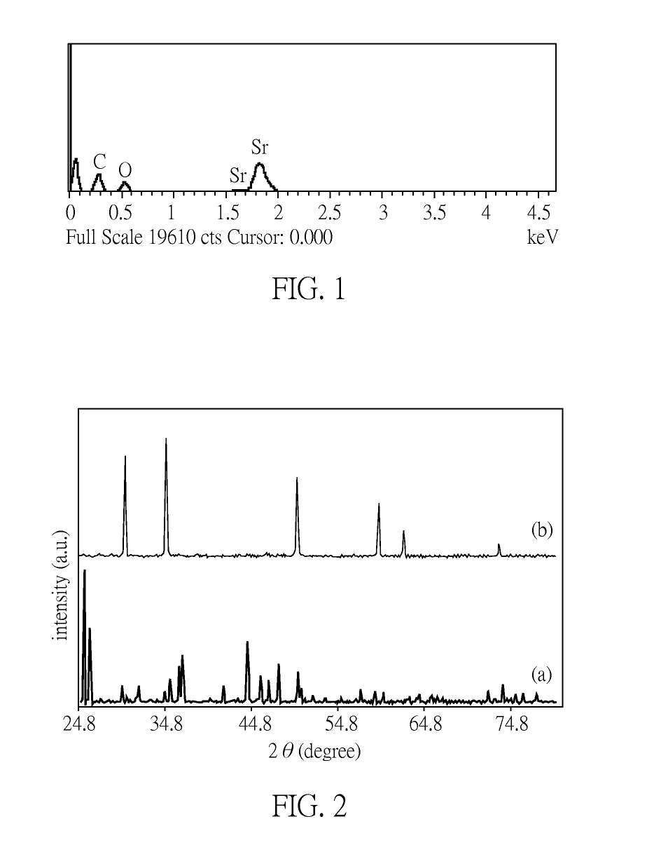

FIG. 2 is an X-ray diffraction (XRD) pattern of the SrO nano-particles adhered on the silica supports formed through the microwave radiation treatment, in which figures (a) and (b) respectively represent the products annealing at 700° and 800° C. It can be seen from the figures that SrCO₃ is the predominant product when heated to 700° C., while SrO is obtained after annealing at 800° C. Accordingly, the annealing process prefers to be performed under 800° C. or more, and more preferably 850° C. or more to reduce the possibility of SrCO₃ formation.

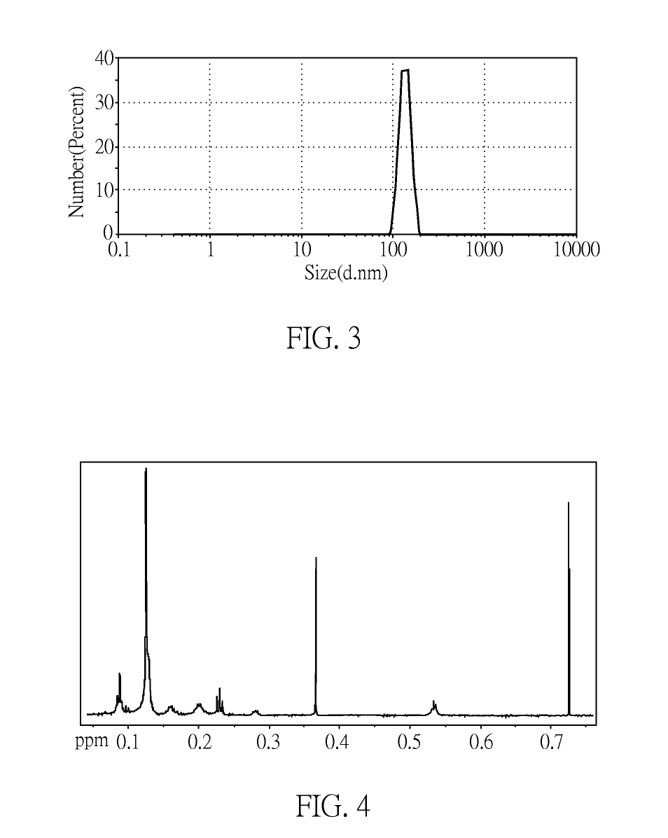

FIG. 3 depicts a diagram showing the size distribution of the SrO nano-particles measured by Dynamic Light Scattering (DLS) analysis, and the average particle size thereof was found to be about 136 nm. This result is consistent with the size measured from the SEM image.

### Transesterification Reaction of the Microalgae

The catalytic effect of the aforementioned SrO nano-particles deposited on the silica supports were examined by performing the transesterification on the Nannochloropsis microalgae in an microwave oven (1200 W), and the Nannochloropsis microalgae is used directly on the dry biomass without extracting the lipidic mass therein. This one step process was conducted in the microwave oven for 2 min. The fatty acid methyl ester (biodiesel) product was dissolved in CDCl₃ and the biodiesel conversion percentage was measured by a 200-MHz ¹H nuclear magnetic resonance (NMR) spectrometer. The biodiesel conversion percentage was calculating by integrating the area under the NMR peaks.

FIG. 4 is a nuclear magnetic resonance (NMR) spectrum of biodiesel. As shown in FIG. 4, no triglyceride peak at the range of 44-35 ppm was observed, and the characteristic peak at 3.65 ppm indicates the methyl group of the methyl ester of fatty acids. The NMR data of FIG. 4 indicate the presence of the strong methyl singlet peak in the biodiesel generated by the method of the present application, and there is no triglyceride contaminated in the generated biodiesel.

The aforementioned result indicates that triglyceride is completely converted into biodiesel within 2 min. The calculated conversion percentage is 99.9% and the percentage of the oil in the microalgae is 37%.

In a similar reaction using commercial micron-sized SrO (obtained from Sigma-Aldrich) instead of the composite containing the SrO nano-particle and the silica support, the reaction was completed in 5 min. Thus, the use of composite of the present application has accelerated the reaction about 2.5 times, compared to the use of the commercial micron-sized SrO. Besides, the separation of the composite to be reused of the present embodiment was much simpler than that of the commercial micron-sized particles.

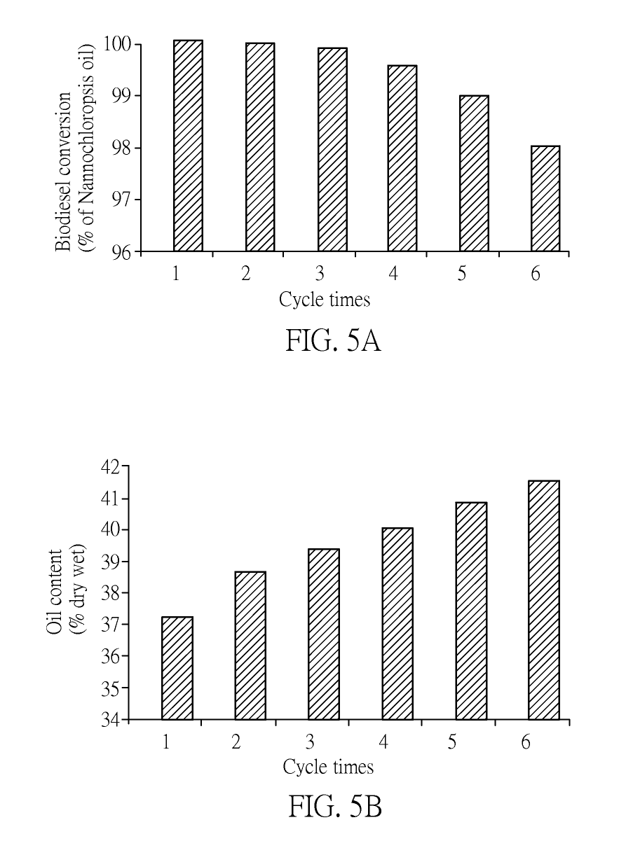

A study for the reuse of the composite containing the SrO nano-particle and the silica support was also conducted. In each cycle, the composites and the residues of the microalgae were separated and the separated composited were added into a new mixture of methanol and microalgae for further another transesterification process. All the reactions were performed for 2 min and the results are shown in FIG. 5A. FIG. 5A demonstrates the stability of the catalytic performance of the composite, and the result indicates an almost constant conversion percentage can be maintained. We also observed a drop of the biodiesel conversion percentage from 99.9 to 97.9% in the 6^{th} cycle.

On the other hand, since we cannot completely removed the residues of the microalgae from the surfaces of the composites, the oil content (%) in the microalgae sample in the 6^{th} cycle was increased from 37% in the first cycle to 41.3%, as shown in FIG. 5B.

### Transesterification Reaction of the Cooked Oil

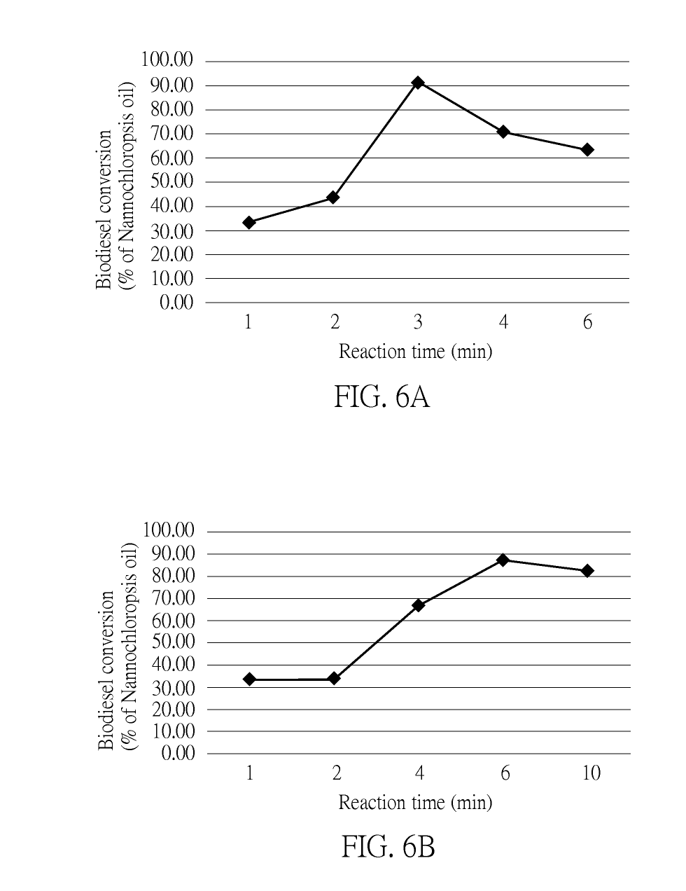

The catalytic effect of the aforementioned SrO nano-particles deposited on the silica supports were examined by performing the transesterification on 2.02 mg KOH/g cooked oil (collected from a recycling location in Taiwan). A mixture was formed of 15 g cooked oil, methanol, and the aforementioned composite in which SrO nano-particles adhered on the silica support under stirring, and then one step process was conducted in a domestic microwave oven (1000 W) respectively for 1, 2, 3, 4, 5, and 6 minutes. The biodiesel (fatty acid methyl esters, FAME) conversion percentage was analyzed using equipment GC-FID HP 6890 with ASTM D6751 and EN14214 methods, and the results is shown in FIG. 6A. Referring to FIG. 6A, the maximum biodiesel conversion percentage from cooked oil is achieved in the 3-min reaction, the calculated biodiesel conversion percentage is about 92% (0.4 mg KOH/g) as well as the percentage of glycerol is about 10% by 0.081 kW power consumption.

Also, in a similar reaction conducted in a domestic microwave oven (700 W), the maximum biodiesel conversion percentage from cooked oil is achieved in the 6-min reaction, as shown in FIG. 6B.

Although the present invention has been explained in relation to its preferred embodiment, it is to be understood that many other possible modifications and variations can be made without departing from the spirit and scope of the invention as hereinafter claimed.

What is claimed is:

1. A method for biodiesel generation, comprising:
providing a plurality of nano-particles each containing an alkali metal compound or an alkaline earth metal compound;
forming a composite in which the nano-particles are adhered on a support; and
performing a transesterification reaction by contacting the composite with a target.

2. The method as claimed in claim 1, wherein each of the nano-particles contains an alkali metal oxide or an alkaline earth metal oxide.

3. The method as claimed in claim 2, wherein the alkaline earth metal oxide is barium oxide (BaO), strontium oxide (SrO), calcium oxide (CaO), or magnesium oxide (MgO).

4. The method as claimed in claim 3, wherein the alkaline earth metal oxide is strontium oxide (SrO).

5. The method as claimed in claim 1, wherein the support is a silicate-based carrier.

6. The method as claimed in claim 5, wherein the support is a silica carrier.

7. The method as claimed in claim 1, wherein a part of the nano-particles are embedded in the support.

8. The method as claimed in claim 7, wherein a second compound is formed between the support and the part of the nano-particles embedded in the support.

9. The method as claimed in claim 1, wherein the support has a size ranging from 1 mm to 30 mm.

10. The method as claimed in claim 1, wherein each of the nano-particles has a size ranging from 20 nm to 1000 nm.

11. The method as claimed in claim 1, wherein an amount of the nano-particles adhered on the support is 0.5 wt % to 10 wt % based on a total weight of the nano-particles and the support.

12. The method as claimed in claim 1, wherein the transesterification reaction is performed through a microwave radiation treatment, a radio frequency treatment, or a laser treatment.

13. The method as claimed in claim 12, wherein the microwave radiation treatment is performed at 60° C. or more.

14. The method as claimed in claim 1, wherein the target is selected from the group consisting of soybean, cottonseed oil, cooked oil, and algae.
